(19) United States
(12) Patent Application Publication
(10) Pub. No.: US 2025/0140418 A1
(43) Pub. Date: May 1, 2025

(54) A COMPUTER-IMPLEMENTED METHOD FOR PROVIDING CARE

(71) Applicant: IESO DIGITAL HEALTH LIMITED, Cambridge (GB)

(72) Inventors: Michael EWBANK, Cambridge (GB); Mihai Valentin TABLAN, Cambridge (GB); Stephanie Ruth BROWN, Cambridge (GB); Dragos GEGEA, Cambridge (GB); Ana Maria Ferreira Paradela Catarino WINGFIELD, Cambridge (GB); Ronan Patrick CUMMINS, Cambridge (GB)

(21) Appl. No.: 18/692,401

(22) PCT Filed: Sep. 15, 2022
(86) PCT No.: PCT/GB2022/052327

§ 371 Date: Mar. 15, 2024

(30) Foreign Application Priority Data
Sep. 15, 2021 (GB) 2113175.0

(51) Int. Cl. G16H 50/30; G16H 10/60; G16H 20/70

(52) U.S. Cl. CPC G16H 50/30; G16H 10/60; G16H 20/70

(57) ABSTRACT

A computer-implemented method for maintaining or improving a user's state of wellbeing, the method comprising: collecting passive data from the user; identifying a cognitive marker within the data and determining a score for the identified marker; predicting the user's state of wellbeing by inputting the score into a statistical predictive model; and generating an output configured to maintain or improve the user's state of wellbeing, wherein the output is based on the user's predicted state of wellbeing.

The present invention relates to computer-implemented methods for providing care and, more specifically, to computer-implemented methods for maintaining or improving a user's state of wellbeing.

The awareness of an individual's state of wellbeing is on the rise. In particular, even healthy people, who have no clinically diagnosable condition, are becoming more concerned with monitoring their current state of wellbeing. Consequently, provisions for predicting and/or determining the changes within an individual's state of wellbeing are of interest. Furthermore, provisions for the early detection of a deteriorating state of wellbeing and/or providing rapid support, coaching, treatment and/or therapy in response to a change in an individual's state of wellbeing are also of interest.

However, such provisions require vast amounts of data as large changes in an individual's state of wellbeing can result from a plurality of very subtle changes within their cognitive and/or behavioral tendencies. In addition, some tendencies can quickly spiral out of control in left unchecked, thus making it increasingly difficult to return an individual to a previous and/or improved state of wellbeing. Monitoring the changes within these tendencies using traditional methods, such as questionnaires and/or surveys, is burdensome on the individual and impractical. Moreover, it is not even possible to accurately capture many of the available data points using questionnaires and/or surveys alone.

Furthermore, in some environments, the state of wellbeing of an individual who has been diagnosed with a mental health condition is not monitored at all during the treatment process. This is partly due to the reluctance of the individual to partake in regular assessments and partly due to the costs and time associated with continuously assessing an individual's state of wellbeing. This can lead to inefficient and/or ineffective treatment plans that may very quickly become unsuitable for an individual whose state of wellbeing has changed.

Meanwhile, the majority of people are continuously creating valuable online data that enables third parties to obtain otherwise difficult, or impossible, to gather information. Therefore, a provision for maintaining or improving a user's state of wellbeing using passive data captured via computer-based systems is of interest.

It is against this background that the present invention has arisen.

According to the present invention there is provided a computer-implemented method for maintaining or improving a user's state of wellbeing, the method comprising: collecting passive data from the user; identifying a cognitive marker within the data and determining a score for the identified marker; predicting the user's state of wellbeing by inputting the score into a statistical predictive model; and generating an output configured to maintain or improve the user's state of wellbeing, wherein the output is based on the user's predicted state of wellbeing.

There is also provided a computer-implemented method for maintaining or improving a user's state of wellbeing, the method comprising receiving data from the user; identifying a feature within the data and determining a score for the identified feature; obtaining a plurality of previous scores corresponding to the identified feature; predicting the user's state of wellbeing based on the determined score and the plurality of previous scores; and sending a notification configured to maintain or improve the user's state of wellbeing, wherein the notification is based on the user's predicted state of wellbeing. The feature may be a marker. The marker may be a cognitive marker.

In some embodiments, the method may comprise generating an output configured to maintain or improve the user's state of wellbeing. The output may be generated instead of sending a notification. The output may be based on the user's predicted state of wellbeing.

These methods may be used to support the early detection of a deteriorating state of wellbeing, such as the presence of an incipient mental health condition, and/or to improve treatments of existing conditions relating to the user's state of wellbeing. In addition, the aforementioned methods may result in less frequent and/or less detailed questionnaires being required in order to monitor a patient's state of wellbeing during a treatment plan. Furthermore, in some scenarios, the need for traditional questionnaires to monitor a patient's state of wellbeing may be entirely unnecessary. This enables an individual's state of wellbeing to be more regularly and accurately monitored, thus enabling a patient's treatment plan to be more frequently reviewed and, if necessary, updated to increase its effectiveness.

The user's state of wellbeing may comprise their physical, mental and/or social wellbeing. Alternatively, or additionally, the user's state of wellbeing may comprise their emotional, psychological, spiritual, educational and/or intellectual wellbeing. More specifically, the user's mental and/or emotional wellbeing may comprise the user's state of stress. Moreover, in some embodiments, the user's state of wellbeing may comprise their mood.

The user's state of wellbeing may be multivariate. For example, the user's state of wellbeing may comprise a plurality of attributes. Attributes of the user's state of wellbeing may comprise at least one of mood, level of anxiety, stress, energy, motivation, and/or engagement. Therefore, in some embodiments, the method comprises predicting at least one attribute of the user's state of wellbeing by inputting the score into the statistical predictive model. This may be instead of, or as well as, predicting the user's (complete) state of wellbeing. The method may comprise predicting a plurality of attributes of the user's state of wellbeing. The output may be based on the at least one predicted attribute of the user's state of wellbeing.

For example, the method may be used to determine if the user is acting in an atypical or uncharacteristic manner that may increase the likelihood of a decreased state of wellbeing. Upon predicting a decreased state of wellbeing, an output is generated in order to maintain or improve the user's state of wellbeing, thus decreasing the likelihood of further deterioration. Alternatively, or in addition, the method may be used to monitor the progress of the user who is seeking to improve their state of wellbeing. In this scenario, the output may be configured to reward and/or encourage the user to continue with their progress.

The output may deliver a care protocol. The care protocol may be a clinical protocol, for example a psychotherapy protocol. The protocol may be a CBT protocol, for example a transdiagnostic CBT protocol.

The output may be configured to provide at least of one of support, coaching, treatment, and/or therapy. The treatment may comprise stress management. The therapy may be psychotherapy. The psychotherapy may be provided as a clinical level intervention. Alternatively, or in addition, the therapy may be preventative therapy. Preventative therapy may be appropriate for an individual previously identified as being at risk of a poor and/or deteriorating state of wellbeing during a pre-clinical phase. For example, the output, when provided in the form of preventative therapy, may be configured to prevent the worsening of the user's symptoms and/or state of wellbeing.

Psychotherapy may comprise preventative therapy, intervention therapy, and/or coaching. For example, the output may provide psychotherapy, this resulting in the user receiving protection, prevention, and/or treatment measures relating to their wellbeing and, in particular, their mental wellbeing. Each of the aforementioned treatment measure may be within the context of care. Psychotherapy may comprise cognitive behavioural therapy (CBT) or acceptance and commitment therapy (ACT).

In some embodiments, the output may provide psychotherapy for treating common mental health conditions, such as depression and/or anxiety disorders. Alternatively, or in addition, the output may provide support to users with long-term physical medical conditions, such as diabetes, chronic obstructive pulmonary disease (COPD), asthma, and/or arthrosis.

In some embodiments, an output may be generated in response to a predicted improvement or positive change in a user's state of wellbeing. The output may be a notification. Alternatively, in some embodiments, the output may be an alert. An alert may be sent in response to a predicted negative change in a user's state of wellbeing. Finally, in some embodiments, an output may be generated to try and maintain a user's state of wellbeing. For example, when the output is a notification, the notification may comprise ‘go for a walk’; ‘take dad for a walk’; ‘well done, you achieved . . . ’.

Alternatively, or in addition, the output may notify a user that their state of wellbeing is lower than their previous state of wellbeing. The output may comprise a suggestion for preventative action against a lowered state of wellbeing, such as an encouragement to engage in an exercise. The exercise may comprise a breathing exercise, meditation, or physical exercise. Physical exercise may comprise going for a walk, run, or playing a sport, for example. However, any form of exercise may be suggested.

In some embodiments, the output may notify a user that they have increased their level of physical activity compared to the previous week. Moreover, when the output is a notification, the notification may send congratulations for doing so. In some embodiments, the output may comprise a reward. The reward may be a physical reward or a digital reward.

In some embodiments, the output is provided to a third party. The third party may be a family member of the user. The third party may be notified that the user's predicted state of wellbeing is decreasing. For example, a third party, such as a family member, may be encouraged to engage in some activity with the user, such as a telephone call, taking them to a movie or going for lunch together.

In some embodiments, wherein the user is a patient of a therapist, the output may comprise sending a notification to the therapist. The notification may present the therapist with a dashboard indicating the state of wellbeing of the user. More specifically, the dashboard may comprise at least one attribute of the user's state of wellbeing. The at least one attribute may comprise the mood, anxiety level, and/or stress level of the user. The therapist may use the output to inform their conversation with the user. Alternatively, or in addition, the dashboard may comprise indicators of behavioural data. The indicators of behavioural data may comprise an increase or decrease in “level of physical activity” or “social connectivity score”.

In some embodiments, the therapist's dashboard may comprise indicators of cognitive data. The indicators of cognitive data may comprise an increases or decreases in occurrences of cognitive distortions like “externalisation of self-worth”, “black and white thinking”, and/or “catastrophising”.

The method comprises collecting passive data from the user and may further comprise collecting active data from the user.

Active data is data that is primarily generated for use within the claimed method. Active data may be provided by the user upon request. To supply active data, the user needs to spend time entering data, coming up with and/or formulating answers. For example, active data comprises text data relating to a therapy session between a therapist and a patient.

Passive data is data that is primarily generated for another purpose, i.e. not for use within the claimed method. Passive data may be collected opportunistically from applications on the user's mobile phone without the involvement and/or knowledge of the user. Passive data generally refers to any data that is not active data. Unlike active data, passive data may be completely objective, for example the number of steps the user has taken in the course of a day. In other embodiments, passive data may be completely subjective, for example the feelings of the user expressed in social media messages. In some embodiments, the passive data collected may have both objective and subjective components.

The method may comprise collecting data from a plurality of sources. The data may be collected via a push and/or pull Application Programming Interface (API). The plurality of sources may comprise at least one of the user's mobile phone, an application installed on the user's mobile phone, the cloud, an external memory device, a questionnaire, a therapy sessions and/or a text-based message. For example, passive data may be collected from internet browsers, websites and/or mobile phones. Conversely, active data may be collected via at least one of a questionnaire, survey, and therapy session.

The data may comprise text data. Alternatively, or in addition, the data may comprise audio data. Audio data may be converted into text data. This may be done using transcription software. The data may also comprise a video or an image, such as an emotion icon or emoticon.

Determining a score for the identified marker may comprise identifying words and/or phrases associated with the marker. This may be done using natural language understand (NLU). The NLU may be a deep-learning-based NLU. In other words, determining a score for the identified marker may comprise determining the meaning of at least a portion of the data. The score may be a frequency. For example, the score may be the number of times a particular meaning is identified. Alternatively, the score may be the percentage of the data that contributes to a particular meaning (i.e. 50% catastrophizing). Alternatively, or in addition, the score may be configured to dictate the probability that the identified meaning is correct (i.e. 50% confident).

The score may be determined using a processor. The processor may be located on the user's device. The statistical predictive model may also be located on the user's device. As such, the entire method may be carried out on the user's device. This may ensure the privacy of the user's data.

Alternatively, the statistical predictive model may be located remote from the user's device. For example, only the determined score may be transferred from the user's device. This may assist with maintaining the user's privacy. Alternatively, the processor and the statistical predictive model may be located within a system that is remote from the user's device and configured to improve and/or marinating the user's state of wellbeing. In such embodiments, raw data may be collected by the remote system.

The statistical predictive model may be built from data from at least one previous user. For example, the statistical predictive model may comprise at least one previous score from at least one previous user. Each previous score may be associated with the identified marker.

The at least one previous user may be the user. In other words, the at least one previous user may be the current user. This enables changes in trends within the user's data over time to be identified and used to predict changes in the user's state of wellbeing.

Alternatively, or in addition, the at least one previous user may be a different user. This enables an output to be generated even if the current user presents a new marker that has only been presented once before by previous user that is different to the current user.

In some embodiments, the at least one previous user is a plurality of previous users. The plurality of previous users may comprise the current user and/or different users. The plurality of previous users may be a group of users that share a particular characteristic. The characteristic may be age, sex, location or any other suitable characteristic. This enables the model to compare the current user's current score against a threshold score calculated from cohort data.

The statistical predictive model may be a deep learning model. The statistical model may comprise multiple user characteristics, including, but not limited to, at least one of age, gender, sex, education, and medical history. A deep learning model provides a reliable method for identifying which marker(s) and corresponding scores have an impact on the user's state of wellbeing. The statistical predictive model may be created using machine learning and data mining to predict and forecast likely future outcomes with the aid of historical and existing data. This works by analysing current and historical data and projecting what it learns on a model generated to forecast likely outcomes.

Alternatively, or in addition, predicting the user's state of wellbeing may comprise calculating the mean and the standard deviation of the previous scores associated with the identified marker; and predicting the user's state of wellbeing based on the determined score and the standard deviations of the previous scores.

This enables the method to determine how different the current score is from the previous scores, which, in turn, allows the computer-implemented method to predict the user's state of wellbeing. For example, the user's state of wellbeing may be predicted based on the difference between the determined score and the mean of the previous scores.

The method may comprise collecting external data. More specifically, the method may further comprise collecting external data; and predicting the user's state of wellbeing based on the external data and the determined score. External data may be obtained from an external system. The external system may be a system that is distinct from a system used by the user (‘user system’). For example, the external system may be a computer-based system for maintaining or improving a user's state of wellbeing and/or a computer-based system capable of carrying out the aforementioned computer-implemented method (‘central system’). The external data may be environmental data. Environmental data may comprise the weather conditions, local temperature, and/or number of daylight hours, for example.

Alternatively, or in addition, the external data may comprise sentiment analysis of news in the user's locality; current level of unemployment; and/or changes in financial markets, for example. Moreover, the external data may comprise a date and/or timestamp. For example, the external data may comprise the time of the year (e.g. to capture proximity of secular and religious festivals). In Western Europe and North America, for example, average mood may be highest just before Christmas, and lowest in January. Conversely, the average mood in Australia may be highest in January due to it being full summer. Any other suitable form of external data may be collected and/or used within the method.

The addition of external data may be used to predict the user's state of wellbeing more accurately. For example, external data acknowledging that it has rained for the previous week may, at least partially, explain why the user has spent more time at home than the week before.

The method may further comprise identifying a behavioural marker within the collected data and determining a score for each identified marker. Predicting the user's state of wellbeing may comprise inputting each score into the statistical predictive model.

Using both cognitive and behavioural markers provides more data points for comparison with the model, thus enabling a more accurate prediction of the user's state of wellbeing. In addition, cognitive and behavioural marker may be independent from one another, thus further increasing the accuracy with which the user's state of wellbeing can be predicted.

The behavioural marker may be at least one of: use of an App, social connectivity score, location, and places visited. Other behavioural markers can also be envisaged. For example, the behavioural marker may be a physical marker. The physical marker may be at least one of: activity, steps, heart rate and sleep pattern. Other physical markers can also be envisaged.

The method may comprise identifying a plurality of cognitive and/or behavioural markers within the data. A score may be determined for each identified marker. Each score may be inputted into the model and, therefore, used to predict the user's state of wellbeing. Identifying a plurality of markers and using the plurality of markers to predict the user's state of wellbeing increases the accuracy with which the user's state of wellbeing can be predicted. For example, a score may be determined for all available markers and all available scores may be used to predict the user's state of wellbeing. This enables the method to account for markers and scores that may cancel each other out. For example, a user who uses social media regularly and gets plenty of exercise may have a better state of wellbeing than a user who also uses social media regularly but does very little exercise.

Of course, any number of markers may be used and/or identified. For example, 1, 2, 3, 4, 5, 6, 7, 8, 9 or 10 markers may be used and/or identified. In some embodiments, more than 10, 20, 30, 50, or 100 features may be used and/or identified.

The method may comprise at least one of: determining the state of wellbeing of the user; and determining the state of wellbeing of at least one previous user. The method may comprise updating the model to comprise at least one of: each determined score; a determined state of wellbeing of the user; and a determined state of wellbeing of at least one previous user. The model may be continuously updated. In other words, the model may be a dynamic model.

The method may further comprise generating a user profile; and updating the user profile following collection of the data from the user, wherein the user profile comprises each of the user's determined scores. The user profile may also comprise at least one user characteristic. The user profile may be used to personalise the statistical predictive model. Personalising the statistical predictive model based on the user profile may be achieved by tuning.

A user profile comprising the determined score provides previous scores against which subsequent determined score may be compared. Continually generating and updating the user profiles each time the method is carried out enables more accurate predictions to be made in the future. Moreover, the user profile may further comprise user characteristics, such as the user's demographics or medical history.

Moreover, in some embodiments, creating and updating the user profile with the determined score enables a baseline to be identified for each marker for each user. This enables the comparison of the user's own previous data and new data from the user, which can significantly improve the precision with which the user state of wellbeing can be predicted.

The user profile may comprise at least one user characteristic. For example, the user profile may comprise the user's name, age, sex, country/location of residence, contact details, family members, symptoms, intensity of symptoms, triggers and/or frequency of input during a text-based conversation, to name a few. However, the previous list is non-exhaustive. Any information regarding the user may be stored within the user profile. The user profile may also comprise at least one previous score from the user.

The method may further comprise determining the user's state of wellbeing and updating the user's profile to comprise the determined state of wellbeing. The determined state of wellbeing may be the current state of wellbeing.

The user state of wellbeing may be determined using a questionnaire or survey. Similarly, attributes of the user's state of wellbeing may be determined using a questionnaire or survey. For example, the user's state of wellbeing, or attributes thereof, may be measured using any suitably validated self-assessment questionnaire, including, but not limited to at least one of a: Patient Health Questionnaire (PHQ-9); Generalised Anxiety Disorder Assessment (GAD-7); and/or Patient Activation Measure (PAM).

The PHQ-9 is a measure of depressive symptoms, ranging from 0 to 27, with higher scores representing greater depression severity. The GAD-7 is a measure of anxiety symptoms, ranging from 0 to 21, with higher scores representing higher anxiety severity.

The PAM questionnaire returns a total score and a PAM level. The total score is a continuous variable, ranging from 0 to 100, with 100 representing maximum patient engagement with the management of their physical health condition. The PAM level is an ordinal variable with 4 levels, with higher levels representing better patient engagement with the management of their physical health condition: Level 1—“Disengaged and Overwhelmed”; Level 2—“Becoming Aware, But still Struggling”, Level 3—“Taking Action”, Level 4—“Maintaining Behaviours and Pushing Further”.

Therefore, determining the user's state of wellbeing, or attributes thereof, using a questionnaire or survey requires active data. Conversely, predicting a user's state of wellbeing may be achieve in the absence of active data. Although, active data may also be used in some instances to predict the user's state of wellbeing. For example, the user's state of wellbeing may be predicted when there is insufficient active data to determine the user's state of wellbeing. Similarly, attributes of the user's state of wellbeing may be predicted when there is insufficient active data to determine the user's state of wellbeing.

The state of wellbeing of the user may be determined at predetermined time intervals. For example, the user may complete a questionnaire or survey each week, month, quarter or year. This may be used to measure the accuracy and/or to re-calibrate the statistical predictive model. Alternatively, or in addition, specific attributes of the user's state of wellbeing may be determined at predetermined time intervals. This may be achieved using partial surveys/questionnaires and/or individual targeted questions.

Within a clinical setting, the state of wellbeing may be determined more frequently. For example, the state of wellbeing may be determined daily or weekly. Alternatively, or in addition, within a consumer setting, the user's state of wellbeing may be determined monthly, quarterly, or yearly, for example.

In some embodiments, the user's profile is continuously updated. For example, the user's state of wellbeing may be determined using a number of partial surveys configured to gather information for a specific aspect of the user's profile. The specific aspect may be an attribute of the user's state of wellbeing and/or a characteristic of the user. ‘Continuously’ may mean each minute, hour, day or week. This may vary over time and from user to user. Consequently, ‘continuously’ may mean ‘at every available opportunity’.

The output may be an action. The action may relate to a treatment protocol. For example, the output may initiate a treatment protocol configured to improve or maintain the user's state of wellbeing. The treatment protocol may be a psychotherapy treatment protocol. The output may be configured to connect the user to a digital psychotherapy platform. The digital psychotherapy platform may be configured to maintain or improve the user's state of wellbeing. The digital psychotherapy platform may put the user in contact with a human clinician or therapist. Alternatively, or in addition, the digital psychotherapy platform may deliver an automated digital psychotherapy protocol. In some embodiments, the output may trigger an automated assessment. The automated assessment may understand the needs of the user. In some embodiments, the output is configured to modify the user's existing treatment protocol.

The output may be based, at least in part, on the identified marker. This may enables the output to deliver the most suitable action and/or information to the recipient in response to an identified score or change in score for a specific marker. For example, a user who has not left the house for multiple days may be encouraged to go and see friends in town.

Alternatively, or in addition, the output may be based, at least in part, on the user's profile. This enables the method to deliver the most suitable output to the recipient in response to an identified score or change in score for a specific marker. For example, a user who has not left the house for several days may typically be encourage to go and see friends in town, as previously described. However, a user known to have social anxiety may be encouraged to spend time in their garden or a local park instead. This may increase the likelihood of maintaining or improving the user's state of wellbeing.

Each previous score may be associated with a state of wellbeing of a previous user. The previous user may be the same user as the current user, or a different user. Alternatively, or in addition, the previous scores may correspond to data collected from a plurality of previous users. The plurality of previous users may be a group of previous users. The group may be defined based on at least one of age, gender, nationality, location, religion, ethnicity, occupation, and level of education. However, other groups may also be defined. A group may comprise any number of previous users. For example, a group may comprise up to 2, 5, 10, 50, 100, 1,000, 10,000, 100,000, 1,000,000, 10,000,000 or more than 10,000,000 previous users.

Using previous scores that correspond to data collected from a plurality of previous users enables the method to be used to predict the state of wellbeing of a new user who has not provided any previous data against which to benchmark the new data.

Using previous scores that correspond to data collected from a plurality of previous users also enables the method to be used to predict the state of wellbeing of an existing user who has provided data comprising a new marker has not been present within the user's previous data. Again, this allows the method to benchmark the collected data against previous users where a benchmark for the current user is not available.

Alternatively, or in addition, the previous scores may correspond to data previously collected from the user. For example, in some embodiments, the statistical predictive model may be built entirely using the current user's previous data. Using data previously collected from the user enables a benchmark score to be calculated for each marker for each user. This enables a much smaller difference in score to be used to predict a change in the user's state of wellbeing. The user's state of wellbeing may therefore be predicted more accurately than using other previous users' previous scores.

The output may comprise a notification. The notification may be sent to at least one of the user and a third party. In some embodiments, the output is sent to a third party. The third party may be a user-defined parameter. The third party may be anybody except the user. The third party may be a family member of the user, relative of the user and/or friend of the user. A family member may be an extended family member. For example, the user's family members may comprise a spouse, partner, sibling, parent, grandparent, grandchild, cousin, aunt, uncle, step-parent or step-sibling. A family member may also be a foster family member or an adopted family member. Alternatively, or in addition, the third party may be a therapist, clinician, carer, or medical professional.

The third party may also be a service provider. The service provider may collect the data. More specifically, the service provider may collect the passive data. The service provider may then provide the data for use within the method. An example service provider may be Meta Platforms, Inc (i.e. the owners of Facebook, Instagram, WhatsApp etc. . . . ). However, any service provider may be used. Therefore, the passive data may comprise data in the form of social media posts, such as Facebook post, Twitter tweets, LinkedIn posts, and/or Instagram captions. However, any social media platform and/or post format may be used to obtain data.

Alternatively, or in addition, the passive data may be collected from instant messages, text-messages, emails, or video calls. Instant messages and/or text-messages may include images, such as emotion icons. Passive data may also be collected from a user's activity tracker; usage history from an app or website; or the user's geolocation.

In a consumer application, the recipient of the output may be the user. For example, when the output is a notification, the notification may be “changes in your behaviour indicate a risk of lower mood. You can improve that by doing more exercise, sleeping better, doing meditation, and using your electronic devices less”.

Conversely, within a clinical setting, the user may be a patient under the care of a clinician. In that case, the notification may be sent to the clinician. The clinician may be a therapist. This may be in addition to, or instead of, sending the notification to the user.

Moreover, the notification sent to the clinician may be different from the notification sent to the user. Therefore, the method may comprise generating a plurality of outputs configured to maintain or improve the user's state of wellbeing. The plurality of output may comprise at least one notification. However, any aforementioned output may be used. Each output may be based on the user's predicted state of wellbeing. For example, the user may receive a notification as previously descried, whereas the clinician may receive a notification comprising information about the trend of the user's mood and/or any other attribute of the user's state of wellbeing.

The data may comprise behavioural data. For example, the behavioural data may be collected from logs from an activity tracker; usage history from an app or website; or the user's geolocation. In some embodiments, the activity tracker may be a FitBit®. The user's geolocation may be their GPS coordinates.

Alternatively, or in addition, the data may comprise cognitive data. For example, the cognitive data may be collected from text generated during a therapy session. The therapy session may be an online therapy session. Alternatively, or in addition, the text may be the transcript of a live therapy session. Using cognitive data generated during a therapy sessions provides data that is otherwise difficult to obtain. This enables the user's state of wellbeing to be predicted more accurately. Cognitive data may also enable a therapist to adapt the content of a therapy session in order to increase the likelihood of improving the user's state of wellbeing. This may be done in real-time and/or in preparation for subsequent therapy sessions. The cognitive data may also be collected from text data generated within a social media application, email, or text-based message. In fact, cognitive and/or behavioural data may be collected from any suitable aforementioned source of data.

The marker may be a behavioural marker. The behavioural marker may be a physical marker. The marker may be an event or occurrence. For example, the marker may be how many people the user has contacted or the size of the user's social network. Alternatively, or in addition, the marker may be a location; the distance travelled during a time period (e.g. a day); the number of distinct locations visited and/or the amount of time spent at home. In some embodiments, the marker may be a heart rate. In some embodiments, the marker may relate to sleep patterns. For example, the marker may be the number and/or duration of general sleep episodes, or, more specifically, at least one of the number and/or duration of awake episodes; the number and/or duration of light sleep episodes; the number and/or duration of rapid eye movement (REM) episodes; and the number and/or duration of deep sleep episodes.

In some embodiments, a marker may be the amount of continuous time (e.g. 8 days) spent at a predetermined location (e.g. at home). Alternatively, or in addition, a marker may be the activation of an App, such as Facebook, within a predefined time period (e.g. the working day, or 9 am-5 pm).

In some embodiments, the marker may be a cognitive marker. A cognitive marker may be an indicator of how somebody (i.e. the user) thinks. The marker may be language associated with deficits in the user's state of wellbeing. For example, the marker may be the type of language (e.g. offensive words), tone (e.g. aggressive or passive), and/or patterns in the use of personal pronouns within a passage of text, or audio clip, generated by the user. Alternatively, or in addition, the marker may be evidence of a cognitive distortion or a cognitive distortion itself. The cognitive distortion may be at least one of “black and white thinking”, “Comparison to Others”, “Fortune-telling”, “catastrophising”, “externalisation of self-worth”, “mind reading”, “Minimisation”, “Overgeneralisation”, “Personalisation”, and “Should Statements”. Descriptions and examples of the aforementioned cognitive distortions are provided in table 1 below. However, this list in non-exhaustive.

TABLE 1
Cognitive distortion categories.

| Category | Description | Examples |
|---|---|---|
| Black and White | Polarised/absolutist thinking; lack of | “There's no going back” |
| Thinking | comprehension of a spectrum of responses | “I can never . . . ” |
| | with only the worst or best outcomes | “I have completely blown it . . . ” |
| | accepted as possible. | “The event wasn't a success, I am a |
| | | complete failure” |
| Comparison to Others | Negative comparison between oneself and | “I'll never be as x as x . . . ” |
| (Unfavourable) | others, belief that one is inferior. | “But they're better than me at . . . ” |
| | | “Everyone there was more x than me.” |
| Externalisation of | Deriving self-worth from how one is viewed | “What I think isn't important” |
| Self-worth | by others. | “I need them to like me.” |
| | | “I just want to fit in” |
| Fortune-telling | Belief in one's own predictions of negative | “It will never get better.” |
| | future outcomes. | “It's going to be a disaster.” |
| | | “They're going to laugh at me” |
| Mind-reading | Belief in arbitrarily reached conclusions | “I knew she thought I was stupid.” |
| | about what others are thinking. | “They hate me.” |
| | | “He didn't say hi to me this morning, it |
| | | must be because he doesn't like me.” |
| Minimisation | Discounting or unreasonably minimising the | “Only a few people laughed when I |
| | positive when evaluating events, | made a joke” |
| | characteristics or actions (glass half empty). | “I passed the test but my score wasn't as |
| | | high as I wanted . . . ” |
| Overgeneralisation | Reaching conclusions either with no | “Last time I went x happened and it will |
| | evidence or by generalising based on limited | happen again” |
| | evidence. | “Everyone will be the same as her” |
| Personalisation | Belief that one is the cause of situations, | “It was my fault that . . . ” |
| | events or others' actions. Lack of | “I was completely responsible for . . . ” |
| | comprehension of other possible causes. | “It was because I made a mistake.” |
| Should Statements | The belief that one should match an | “I should have got a higher test score” |
| | arbitrary internal or external standard. | “I can't make any mistakes” |
| | Unreasonable expectations of oneself | “I need to be perfect” |
| | without consideration of circumstances. | |

The score may be non-binary. For example, the score may be a tally or count of the number of times a specific marker is present within the data. The score may be any numerical value, probability, or frequency.

Alternatively, in some embodiments, the score may be binary. For example, the score may be ‘yes’ or ‘no’. In some embodiments, the score for a first marker may be binary and the score for a second marker may be non-binary. Any number of markers having binary and/or non-binary scores may be used.

According to the present invention, there is also provided a computer-based system for carrying out the aforementioned method.

More specifically, there is provided a computer-based system for maintaining or improving a user's state of wellbeing, the system comprising a data collection module configured to collect passive data from the user; a memory configured to store a statistical predictive model; a processor configured to: identify a cognitive marker within the data; determine a score for the identified marker; and predict the user's state of wellbeing by inputting the score into the statistical predictive model; and an output module configured to generate an output configured to maintain or improve the user's state of wellbeing, wherein the output is based on the user's predicted state of wellbeing.

Each feature of the computer-based system may be located on a device belonging to the user. The device belonging to the user may be a portable, or non-portable, electronic device, such as a mobile phone, tablet, laptop, or computer. Alternatively, some, or all, of the aforementioned features of the computer-based system may be located remotely from the user's device.

The processor may be a single processor or a plurality of processors. At least one processor may be located on a device belonging to the user. The processor on the user's device may determine the score. The score may then be provided to an external processor. The data collection module may be further configured to receive external data. The data collection module may comprise a push and/or pull application programming interface (API). The data collection module may also be located on the user's device. The processor may be configured to predict the user's state of wellbeing by inputting the external data and the score into the statistical predictive model. The processor may be an arithmetic logic unit (ALU).

The memory may comprise a user data module configured to store a plurality of user profiles each having information about the corresponding user. The user information may comprise at least one of the aforementioned user characteristics. Each user profile may comprise a determined state of wellbeing of the corresponding user. The plurality of user profiles may comprise the current user's profile and/or previous user's profiles. Additionally, or alternatively, each user profile may comprise one or more determined states of wellbeing of the corresponding user. Each state of wellbeing stored within a user profile may also be associated with a timestamp. Alternatively, or in addition, each user profile may comprise at least one score corresponding to at least one marker. The marker may be the identified cognitive marker.

The statistical predictive model may be a dynamic model configured to update continuously such that it comprises: each determined score; a newly determined state of wellbeing of the user; and/or a newly determined state of wellbeing of at least one previous user.

The invention will now be further and more particularly described, by way of example only, with reference to the accompanying drawings, in which:

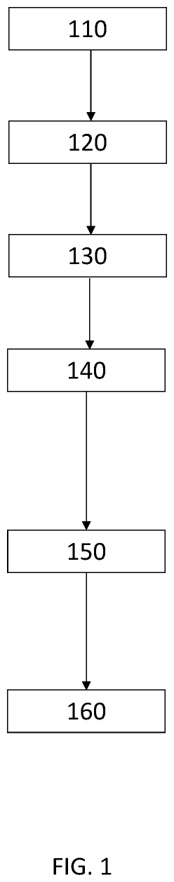

FIG. 1 shows a computer-implemented method for maintaining or improving a user's state of wellbeing according to some embodiments of the invention;

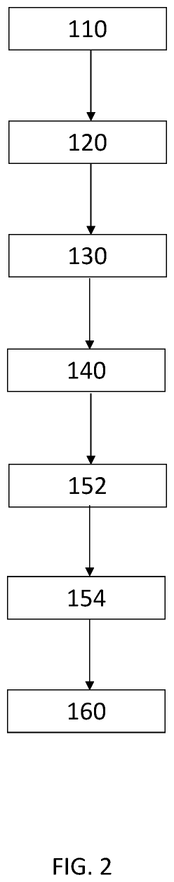

FIG. 2 shows the computer-implemented method of FIG. 1, wherein the user's state of wellbeing is predicted based on the determined score and the standard deviation of the previous scores;

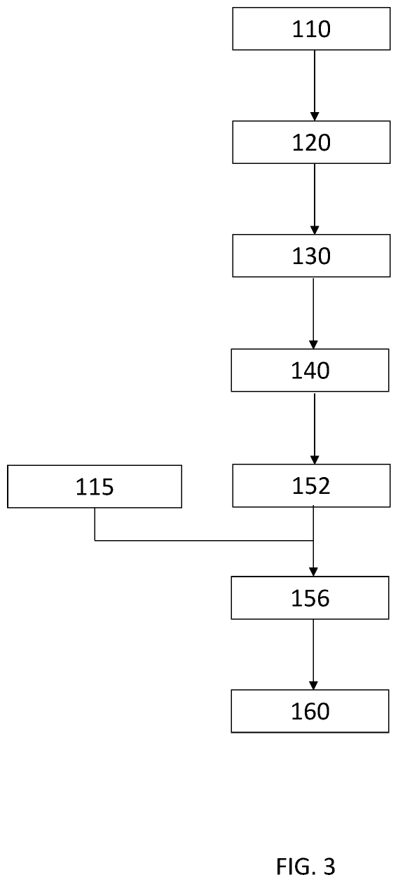

FIG. 3 shows the computer-implemented method of FIG. 2, wherein the user's state of wellbeing is predicted based, at least in part, on external data;

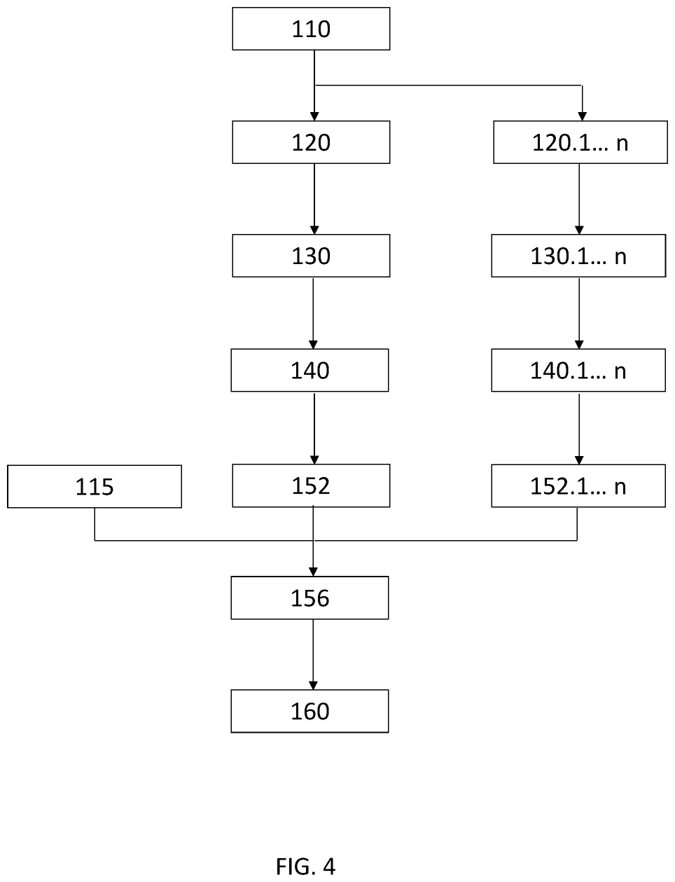

FIG. 4 shows a computer-implemented method for maintaining or improving a user's state of wellbeing, wherein the user's state of wellbeing is predicted based, at least in part, on a plurality of features within the user's data;

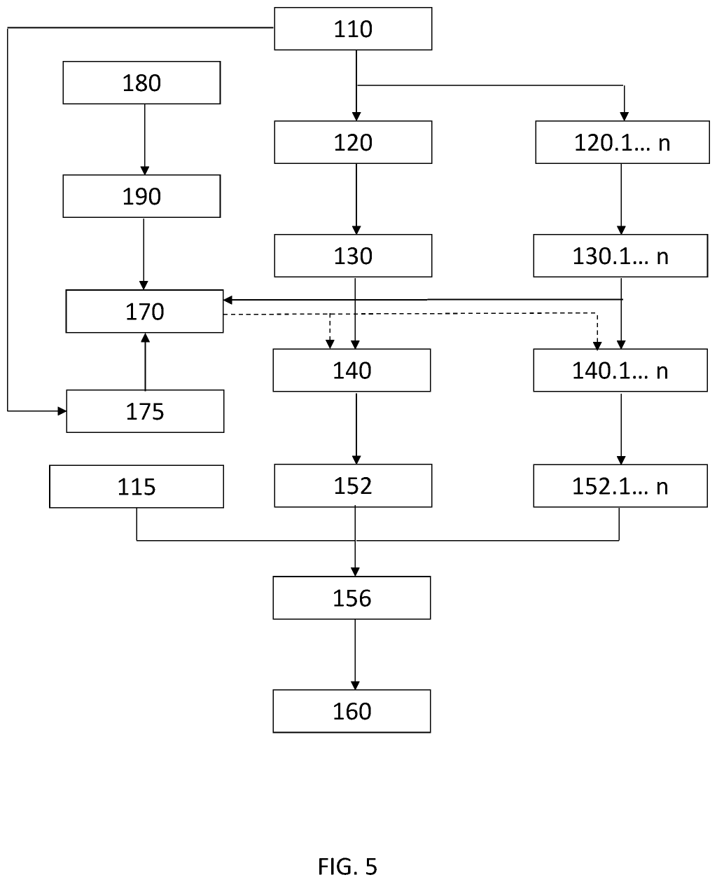

FIG. 5 shows the computer-implemented method of FIG. 4, further comprising the generation, maintenance and use of the user's profile for predicting their state of wellbeing;

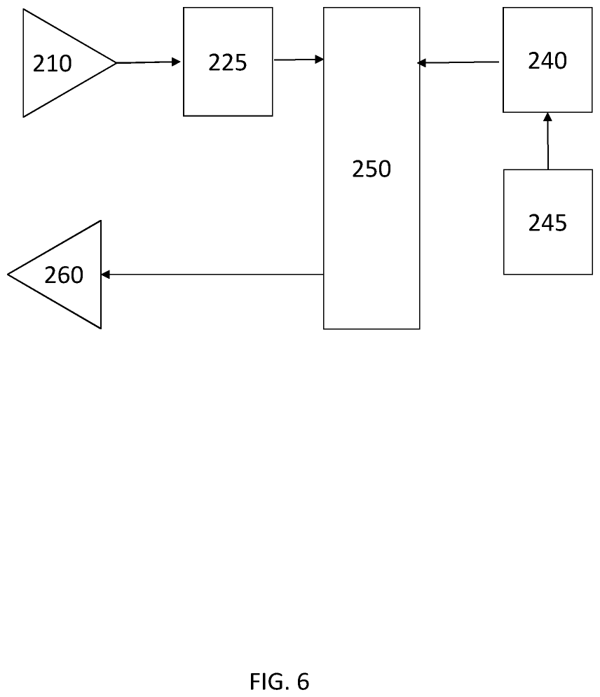

FIG. 6 shows a computer-based system for maintaining or improving a user's state of wellbeing according to some embodiments of the invention;

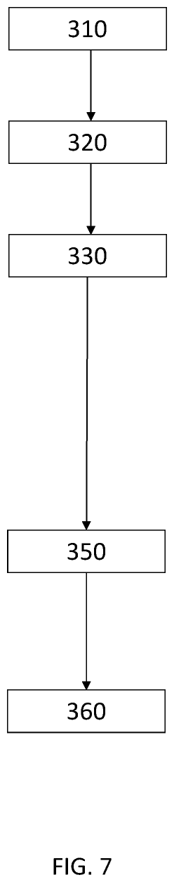

FIG. 7 shows a computer-implemented method for maintaining or improving a user's state of wellbeing according to some embodiments of the invention;

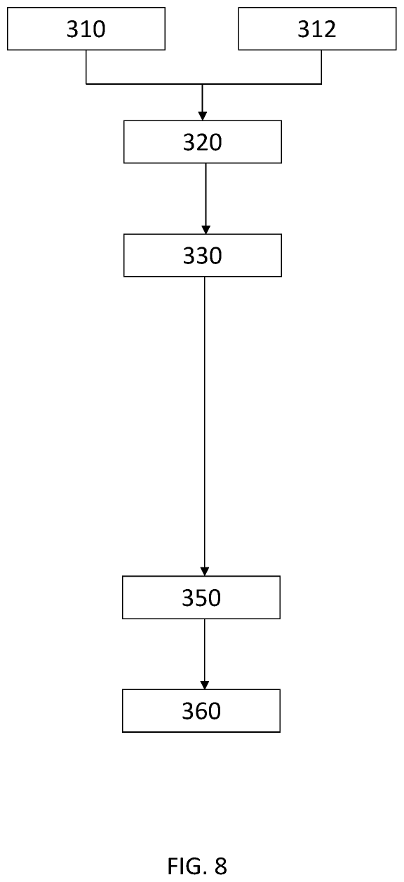

FIG. 8 shows the computer-implemented method of FIG. 7, further comprising the step of collecting active data from the user;

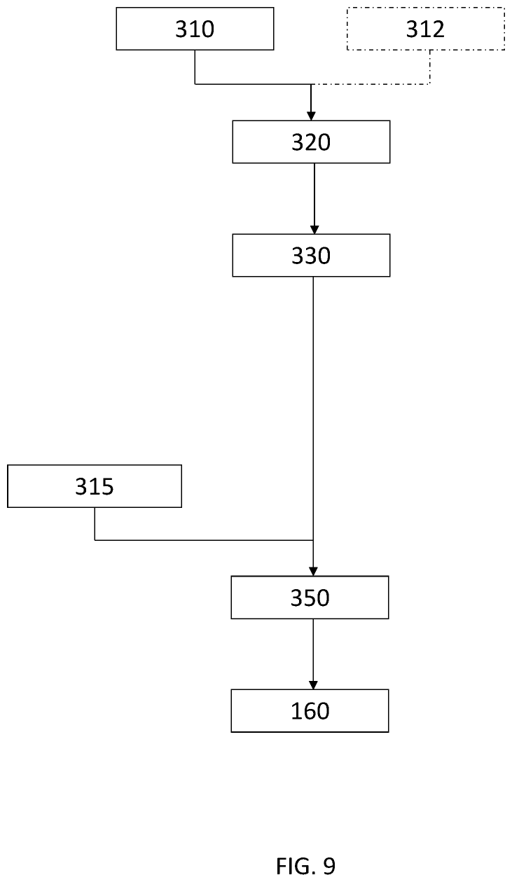

FIG. 9 shows the computer-implemented method of FIG. 7 or 8, wherein the user's state of wellbeing is predicted based, at least in part, on external data;

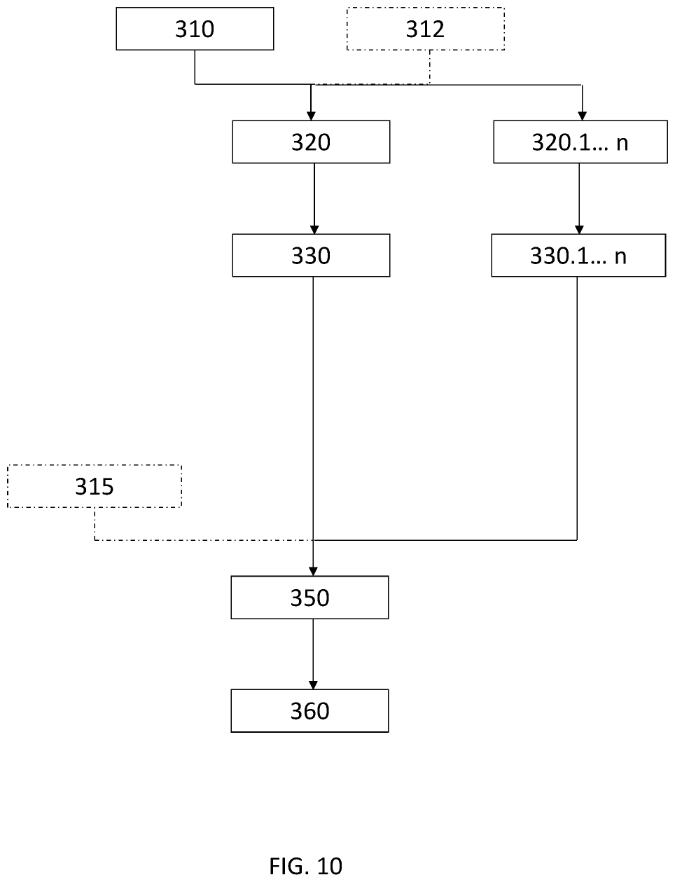

FIG. 10 shows the computer-implemented method of any of FIGS. 7 to 9, wherein the user's state of wellbeing is predicted based, at least in part, on a plurality of markers within the user's data;

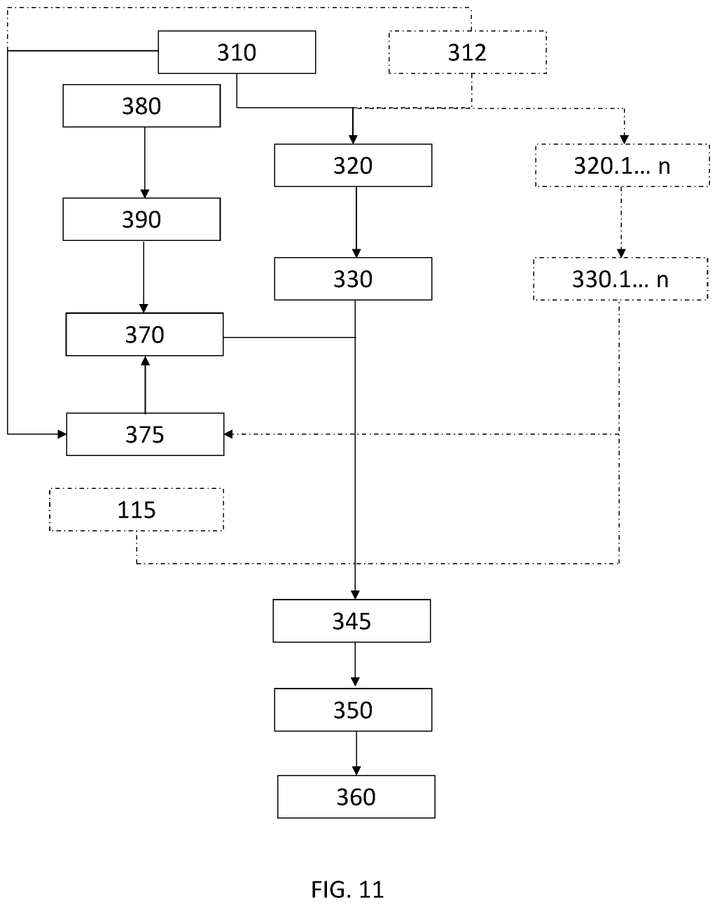

FIG. 11 shows the computer-implemented method of any of FIGS. 7 to 10, further comprising the generation, maintenance and use of a user profile; and

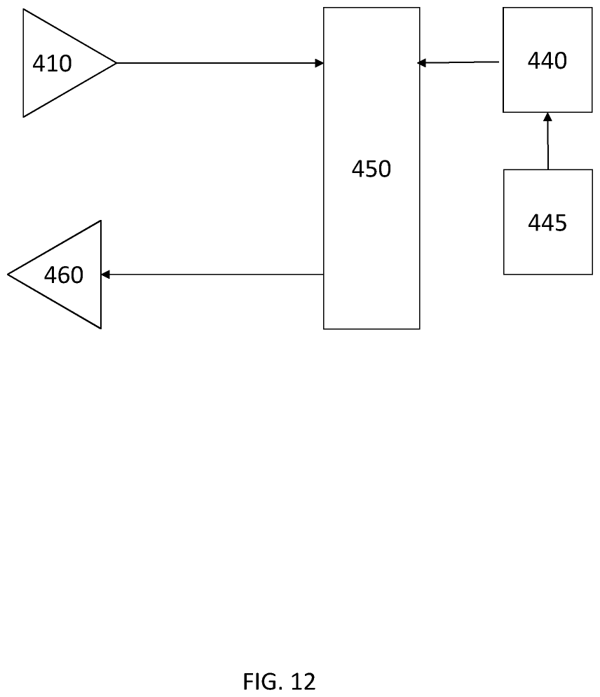

FIG. 12 shows a computer-based system for maintaining or improving a user's state of wellbeing according to some embodiments of the invention.

Throughout the following description, like reference numerals are used to describe equivalent steps in each method.

FIG. 1 shows a computer-implemented method for maintaining or improving a user's state of wellbeing according to some embodiments of the invention. The method comprises the steps of receiving 110 data from the user; identifying 120 at least one feature within the data; determining 130 a score for the identified feature or each identified feature; obtaining 140 a plurality of previous scores corresponding to the identified feature or each identified feature; predicting 150 the user's state of wellbeing based on the determined score or scores and the previous scores; and sending 160 a notification configured to maintain or improve the user's state of wellbeing. The notification is based on the user's predicted state of wellbeing.

FIG. 2 shows the computer-implemented method of FIG. 1, wherein the user's state of wellbeing is predicted based on the determined score and the standard deviation of the previous scores. More specifically, once the plurality of previous scores corresponding to the identified feature have been obtained 140, the method comprises the additional steps of calculating 152 the mean and the standard deviation of the plurality of previous scores corresponding to the identified feature; and predicting 154 the user's state of wellbeing based on the determined score and the standard deviation of the previous scores.

FIG. 3 shows the computer-implemented method of FIG. 2, wherein the user's state of wellbeing is predicted based, at least in part, on external data. More specifically, the method further comprises the steps of receiving 115 external data; and predicting 156 the user's state of wellbeing based on the external data, the determined score and the standard deviation of the previous scores. The external data will be processed in a similar manner to the user's data in that features will be determined within the data and scores will be determined corresponding to the identified feature.

FIG. 4 shows a computer-implemented method for maintaining or improving a user's state of wellbeing, wherein the user's state of wellbeing is predicted based, at least in part, on a plurality of features within the user's data. The method comprises receiving 110 data from the user; identifying 120, 120.1 . . . n a plurality of features within the data; determining 130, 130.1 . . . n a score for each identified feature; obtaining 140, 140.1 . . . n a plurality of previous scores corresponding to each identified feature; calculating 152, 152.1 . . . n the mean and the standard deviation of the plurality of previous scores corresponding to each identified feature; receiving 115 external data which may be processed to identify one or more features within the data and then to determine a score for each identified feature; predicting 156 the user's state of wellbeing based on the external data, the determined score and the standard deviation of the previous scores; and sending 160 a notification configured to maintain or improve the user's state of wellbeing. The notification is based on the user's predicted state of wellbeing.

FIG. 5 shows the computer-implemented method of FIG. 4, further comprising the generation, maintenance and use of the user's profile for predicting their state of wellbeing. More specifically, the method of FIG. 4 further comprises the steps of generating 170 a user profile; determining 180 the current state of wellbeing of the user; updating 190 the user's profile to comprise the current state of wellbeing; receiving 110 data from the user; updating 175 the user profile following receipt of data from the user; identifying 120, 120.1 . . . n a plurality of feature within the data; determining 130, 130.1 . . . n a score for each identified feature; obtaining 140, 140.1 . . . n a plurality of previous scores corresponding to each identified feature; calculating 152, 152.1 . . . n the mean and the standard deviation of the plurality of previous scores corresponding to each identified feature; receiving 115 external data which may be processed to identify one or more features within the data and then to determine a score for each identified feature; predicting 156 the user's state of wellbeing based on the external data, the determined score and the standard deviation of the previous scores; and sending 160 a notification configured to maintain or improve the user's state of wellbeing.

FIG. 6 shows a computer-based system for maintaining or improving a user's state of wellbeing. The system comprises an input module 210 configured to receive data from the user; a processor 225 configured to identify a feature within the user data and determine a score for the identified feature; a memory 240 configured to store a plurality of previous scores corresponding to the identified feature; an arithmetic logic unit 250 configured to predict the user's state of wellbeing based on the determined score and the plurality of previous scores; and an output module 260 configured to send a notification configured to maintain or improve the user's state of wellbeing. The notification is sent to at least one of the user and a third party. The notification is based on the user's predicted state of wellbeing. The system further comprises a user data module 245 configured to store a plurality of user profiles each having information about the corresponding user.

FIG. 7 shows a computer-implemented method for maintaining or improving a user's state of wellbeing according to some embodiments of the invention. The method comprises the steps of collecting 310 passive data from the user; identifying 320 at least one cognitive marker within the data; determining 330 a score for the identified marker or each identified marker; predicting 350 the user's state of wellbeing by inputting each score into a statistical predictive model; and generating 360 an output configured to maintain or improve the user's state of wellbeing. The output is based on the user's predicted state of wellbeing. In some embodiments, the step 320 further comprises identifying at least one behavioural marker within the collected data. In such embodiments, a score may be determined for each identified behavioural marker. Each determined score (both for cognitive and behavioural markers) may then be inputted into the model and used to predict the user's state of wellbeing.

FIG. 8 shows the computer-implemented method of FIG. 7, further comprising the step of collecting active data from the user. More specifically, the method comprises the steps of collecting 310 passive data from the user; collecting 312 active data from the user; identifying 320 at least one cognitive marker within the data; determining 330 a score for the identified marker or each identified marker; predicting 350 the user's state of wellbeing by inputting each score into a statistical predictive model; and generating 360 an output configured to maintain or improve the user's state of wellbeing. The marker may be identified within the passive data and/or the active data. Preferably, a plurality of markers is identified, wherein at least one marker is identified within the passive data and at least one marker is identified in the active data. Therefore, in some embodiments, the output may be based, at least in part, on the active data. More specifically, the output may be based, at least in part, on a cognitive marker identified within the passive data.

FIG. 9 shows the computer-implemented method of FIG. 7 or 8, wherein the user's state of wellbeing is predicted based, at least in part, on external data. More specifically, the method further comprises the steps of receiving 315 external data; and predicting 350 the user's state of wellbeing by inputting the external data and each score into a statistical predictive model. The raw external data may be inputted into the model. Alternatively, the external data may be processed in a similar manner to the user's data in that at least one marker may be determined within the data and scores may be determined corresponding to each identified marker. In fact, the external data may be processed in any suitable way. The method may or may not comprise collecting 312 active data, which is shown by the dashed lines.

FIG. 10 shows the computer-implemented method of any of FIGS. 7 to 9, wherein the user's state of wellbeing is predicted based, at least in part, on a plurality of markers within the user's data. The plurality of markers comprises at least one cognitive marker. Subsequent markers may be cognitive markers and/or behavioral markers. The method comprises collecting 310 passive data from the user; identifying 320, 320.1 . . . n a plurality of markers within the collected data; determining 330, 330.1 . . . n a score for each identified marker; predicting 350 the user's state of wellbeing by inputting each score into a statistical predictive model; and sending 160 an output configured to maintain or improve the user's state of wellbeing. The output is based on the user's predicted state of wellbeing. The method may or may not comprise collecting 312 active data and/or collecting 315 external data, which is shown by the dashed lines.

FIG. 11 shows the computer-implemented method of any of FIGS. 7 to 10, further comprising the generation, maintenance and use of a user profile. More specifically, the method further comprises the steps of generating 370 a user profile; determining 380 the state of wellbeing of the user; updating 390 the user's profile to comprise the determined state of wellbeing; updating 375 the user profile following collection of data from the user such that the user profile comprises each of the user's determined scores; personalising 345 the statistical predictive model based on the user profile; and predicting 350 the user's state of wellbeing by inputting each score and the user profile into the statistical predictive model.

FIG. 12 shows a computer-based system for maintaining or improving a user's state of wellbeing. The system comprises a data collection module 410 configured to collect passive data from the user; a memory 440 configured to store a statistical predictive model; a processor 450 configured to: identify at least one cognitive marker within the data; determine a score for each identified marker; and predict the user's state of wellbeing by inputting the score into the statistical predictive model; and an output module 460 configured to generate an output configured to maintain or improve the user's state of wellbeing.

The data collection module 410 may be further configured to collect active data and/or external data. The output may be sent to at least one of the user and a third party. The output is based on the user's predicted state of wellbeing. The system further comprises a user data module 445 configured to store a plurality of user profiles each having information about the corresponding user. The user data module 445 may be used to update the statistical predictive model stored within the memory 440.

In some embodiments, the present invention may be used within a consumer wellbeing app. For example, a user may download the app, e.g. from the app store or play store, and start it for the first time. During the set up process, the app may obtain basic demographic data from the user, including their sex, age, self-reported level of physical activity, self-reported level of social engagement. Finally, the user may give the app access to location data, app usage data, health and/or physical activity data. As the user is a new one, the statistical predictive model may initially use population-wide statistics, based on cohorts of previous users with similar demographics and/or self-reported characteristics.

Despite having described themselves as a moderately active person, the app may detect during the first week of use that the user's level of physical activity is 12.4 minutes/day. This is 3.2 standard deviations from the mean of 43.1 minutes/day of other people who consider themselves moderately active. The app may notify the user that their level is physical activity is currently reduced from its normal level, and that reduced physical activity is known to correlate with decreases in mood, and higher stress and anxiety levels. The app may suggest to the user that they plan some activity at the weekend, such as going for a run.

After several months of use, the app may have created a detailed personal profile of the user. A deep neural network may predict that the user's mood has decreased by more than one standard deviation over the last two weeks. While the model uses all available features (physical activity, screen time, number of locations visited per day) to make that prediction, it may find that the two features that contribute most to the output are “use of social media apps” and “screen time”. It may notify the user that their behaviour seems to have changed over the course of the previous two weeks, and that increased use of devices can be detrimental to mental wellbeing. It may encourage the user to plan a “in real life” activity with their friends.

Alternatively, or in addition, the present invention may be used within a care companion app. For example, the user may be a patient being treated for generalised anxiety. As part of their treatment, the user may receive access to the care companion app, which they download, install, and give the appropriate permissions to. The user may log in to the app using their patient account details and password that they created when they started treatment.

During the first week of use, the app may find that the sleep fractionation score for this user is higher than the average for their age group. It may create a notification for the user, explaining the links between sleep and anxiety. The notification may comprise a link to educational material on how to improve one's sleep.

The app may also add the data about the user's sleep score to the server and/or statistical predictive model, as well as a note about the recommendation given to the user. That data may be used to populate a clinician's dashboard that the user's therapist will consult prior to the user's next session. During the therapy session, the therapist may ask the patient whether they read the sleep educational materials. The therapist may also ask whether the user has started making any of the changes suggested therein, such as reducing electronic device use prior to going to bed.

In a study conducted by the applicant, a deep learning model was trained to categorise cognitive distortions expressed by ˜35,000 patients receiving internet-enabled CBT for depression or generalized anxiety disorder. It was found that the quantity and pattern of distortions differed between the two disorders and that distortions showed a differential relationship with symptoms of depression and anxiety. Dichotomous thinking was predictive of poorer outcomes for both disorders, indicating that certain cognitive constructs may be associated with a poorer response to treatment in CBT.

In the study, nine categories of cognitive distortions were identified. These categories are shown above in table 1. The categories were based primarily on the 17 categories defined by Yurica and DiTomasso (2005). Annotators reviewed example transcripts in order to gain an understanding of how the 17 categories defined by Yurica and DiTomasso (2005) might present in patient language during a psychotherapy session and how easily distinguishable they were. These categories were then refined down to the nine categories that were deemed both possible to identify in patient language and that did not overlap with other categories. For example, catastrophising, absolutist, and all or nothing thinking were combined into a single category of ‘Black and White Thinking’. ‘Emotional Reasoning’ was not included as a category as it was found that these statements tended to overlap, or were easily confused, with a patients' descriptions of their symptoms elicited by therapists in the first treatment session (e.g. “I feel unloved”, “I feel scared”).

A set of 140 patient transcripts were then used to train a deep learning model. The trained model was then run over the entire corpus of CBT treatment session transcripts, categorising utterances as either a cognitive distortion, as per the defined categories, or ‘other’ if no distortion was detected. A deep learning model architecture, as described in Cummins et al., (2019), and Ewbank et al., (2019), was used to automatically classify each patient utterance into one or more of the nine cognitive distortion categories previously outlined.

The deep learning model provided output data comprising the count of individual distortion utterances expressed by the patient in each appointment session. An ‘utterance’ was defined for the model as units of speech that typically perform one function or one turn in the synchronous therapy session. For each patient, the number of distortion utterances, as a proportion of all utterances, was calculated for each different type of distortion for the first treatment session only.

All analyses were performed in R (R Core Team, 2017). The original dataset comprised a total of 44729 patients. However, only patients who had completed treatment and ‘engaged’ were included in the analysis. A patient was classed as ‘engaged’ if they attended two or more treatment sessions.

This is the minimum amount of therapy a patient must receive such that pre- and post-treatment measures can be collected and clinical outcomes estimated. Cases with missing start or end PHQ-9 or GAD-7 scores were excluded from the analysis. This left a total of 35344 patients included in the analysis.

A multiple linear regression predicting the total proportion of distortion utterances in the first treatment session revealed that patients with depression showed a significantly greater proportion of distortions than patients with GAD (z=−4.36; p<0.001; depression, estimated marginal mean (EMM)=20.1; GAD, EMM=19.0).

Multivariable linear regression analyses revealed that patients with depression showed a higher proportion of utterances for seven of the nine distortions (Comparison to Others, z=−8.67, p=<0.001; Externalisation of Self-worth, z=−20.37, p=<0.001; Mind-reading, z=−13.84, p=<0.001; Overgeneralisation, z=−10.09, p=<0.001; Personalisation, z=−22.86, p=<0.001; Should Statements, z=−21.30, p=<0.001). Compared to patients with depression, patients with GAD had a higher proportion of Black and White Thinking (z=11.69, p=<0.001) and Fortune-telling (z=44.02, p=<0.001).

A multivariable linear regression for GAD patients only revealed a significant relationship between patients' start GAD-7 score and both Black and White Thinking (z=3.10, p=0.002) and Fortune-telling (z=5.14, p=<0.001). This indicates that the greater the number of anxiety symptoms reported by the patient the greater the proportion of these types of cognitive distortion were present in their first treatment session. By contrast, there was a negative relationship between start GAD-7 scores and Personalisation (z=−2.34, p=0.019).

An analogous multivariable linear regression for depression patients also only revealed a significant positive relationship between start PHQ-9 score and Black and White Thinking (z=3.47, p=0.001) as well as between start PHQ-9 score and Overgeneralisation (z=2.12, p=0.034) indicating that the higher a patient's PHQ-9 score the greater the proportion of these types of cognitive distortions were present in their first treatment session. Fortune-telling (z=−7.76, p=<0.001) showed a negative significant relationship, indicating that the lower the patient's PHQ-9 score the higher the proportion of these distortions in the first treatment session.

A stepwise regression analyses was also used to determine which of the different categories of cognitive distortion in the first treatment session were predictive of symptom scores at the end of treatment, while adjusting for starting symptom scores. For GAD patients, it was found that only three distortions were retained in the final model. Black and White Thinking showed a significant positive relationship with end GAD-7 score (z=2.96, p=0.003), indicating that the greater the proportion of black and white thinking in the first session the higher the scores at the end of treatment (i.e. poorer outcomes). By contrast, Should Statements (z=−2.36, p=0.018) showed a significant negative relationship, and Fortune telling a marginally non-significant negative relationship (z=−1.88, p=0.06) with end GAD-7 score, indicating a greater decrease in scores at the end of treatment (i.e. better outcomes).

An analogous stepwise regression analysis predicting PHQ-9 scores at the end of treatment only in patients with depression revealed that again, only Black and White Thinking was associated with higher scores at the end of treatment (z=3.75, p=<0.001) (i.e. poorer outcomes). Should Statements (z=−5.00, p=<0.001) and Fortune-telling (z=−3.22, p=0.001) showed a significant negative relationship with PHQ-9 scores at the end of treatment (i.e. lower end scores, better outcomes).

Whilst the aforementioned study was carried out using online CBT transcripts, the techniques used therein are equally applicable to any form of text-based message. This includes text-based messages found within active and/or passive data.

Various further aspects and embodiments of the present invention will be apparent to those skilled in the art in view of the present disclosure. “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments that are described. It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1.-23. (canceled)

24. A computer-implemented method for maintaining or improving a user's state of wellbeing, the method comprising:
collecting passive data from the user;
identifying a cognitive marker within the data and determining a score for the identified marker;
predicting the user's state of wellbeing by inputting the score into a statistical predictive model; and
generating an output configured to maintain or improve the user's state of wellbeing, wherein the output is based on the user's predicted state of wellbeing.

25. The method according to claim 1, further comprising: collecting active data from the user.

26. The method according to claim 1, further comprising: collecting data from a plurality of sources.

27. The method according to claim 1, wherein the statistical predictive model comprises at least one previous score from a plurality of previous users, and wherein each previous score is associated with the identified marker.

28. The method according to claim 1, wherein the statistical predictive model comprises at least one previous score from the user, and wherein each previous score is associated with the identified marker.

29. The method according to claim 1, wherein the statistical predictive model is a deep learning model.

30. The method according to claim 1, further comprising:
collecting external data; and
predicting the user's state of wellbeing based on the external data and the determined score.

31. The method according to claim 1, further comprising:
identifying a behavioural marker within the collected data and determining a score for each identified marker; and
predicting the user's state of wellbeing by inputting each score into the statistical predictive model.

32. The method according to claim 8, comprising identifying at least one of a plurality of cognitive and behavioural markers within the data.

33. The method according to claim 1, further comprising:
updating the model to comprise at least one of:
each determined score;
a determined state of wellbeing of the user; and
a determined state of wellbeing of at least one previous user.

34. The method according to claim 1, further comprising:
generating a user profile;
updating the user profile following collection of the data from the user, wherein the user profile comprises each of the user's determined scores; and
personalising the statistical predictive model based on the user profile.

35. The method according to claim 11, further comprising:
determining the user's state of wellbeing; and
updating the user's profile to comprise the determined state of wellbeing;
wherein the output is based, at least in part, on the user's profile.

36. The method according to claim 1, wherein the output is based, at least in part, on the identified marker.

37. The method according to claim 1, wherein the output comprises sending a notification to at least one of the user and a third party.

38. The method according to claim 1, wherein the marker is a cognitive distortion.

39. A computer-based system for maintaining or improving a user's state of wellbeing, the system comprising:
a data collection module configured to collect passive data from the user;
a memory configured to store a statistical predictive model;
a processor configured to:
identify a cognitive marker within the data;
determine a score for the identified marker; and
predict the user's state of wellbeing by inputting the score into the statistical predictive model; and
an output module configured to generate an output configured to maintain or improve the user's state of wellbeing, wherein the output is based on the user's predicted state of wellbeing.

40. The computer-based system according to claim 16, wherein the data collection module is further configured to collect external data.

41. The computer-based system according to claim 17, wherein the processor is configured to predict the user's state of wellbeing by inputting the external data and the score into the statistical predictive model.

42. The computer-based system according to claim 16, wherein the memory comprises a user data module configured to store a plurality of user profiles each having information about the corresponding user; and
wherein each user profile comprises at least one of:
a determined state of wellbeing of the corresponding user; and
at least one score corresponding to at least one marker.

43. The computer-based system according to claim 16, wherein the statistical predictive model is a dynamic model configured to update continuously such that it comprises at least one of:
each determined score;
a newly determined state of wellbeing of the user; and
a newly determined state of wellbeing of at least one previous user.
